(19) United States
(12) Patent Application Publication
(10) Pub. No.: US 2026/0354947 A1
(43) Pub. Date: Nov. 12, 2026

(54) INTRAORAL SCANNING FOR IMPLANT PROSTHODONTICS

(71) Applicant: Dental Imaging Technologies Corporation, Quakertown, PA (US)

(72) Inventors: Victor C. Wong, Pittsford, NY (US); Xiaodong Tao, Pittsford, NY (US); Levon O. Vogelsang, Webster, NY (US)

(73) Assignee: Dental Imaging Technologies Corporation, Quakertown, PA (US)

(21) Appl. No.: 19/201,394

(22) Filed: May 7, 2025

(51) Int. Cl. A61C 13/00; A61B 5/00; A61B 8/12; A61C 9/00; A61C 13/34; G06F 30/10

(52) U.S. Cl. CPC A61C 13/0004; A61B 5/0095; A61B 8/12; A61C 9/0053; A61C 13/34; G06F 30/10

(57) ABSTRACT

A method for dental implant prosthesis design scans a dental arch having a seated implant with a hand-held intraoral scanner and generates a 3-D surface model. Acquired scan data from the intraoral scanner includes both exterior scan data that characterizes visible external surfaces of the seated implant, and interior scan data that characterizes inner surfaces within a hollow shaft that extends into the implant from an outer opening that is visible on the external surface of the implant to an internal base that terminates the shaft. Scanned inner surfaces define the orientation of an axis that extends through the shaft. Angular orientation and position of the implant within the 3-D surface model are identified according to the defined axis defined axis. A virtual digital abutment is mapped onto the implant to form a modified 3-D surface model. The modified 3-D surface model is conveyed to a prosthesis design facility.

### FIELD OF THE INVENTION

The present application is directed to implant prosthodontics and more particularly to methods for modeling implant geometry using intraoral scanning devices and techniques.

### BACKGROUND OF THE INVENTION

Hand-held scanners have brought about significant benefits to prosthetic dentistry. Various Intra-Oral Scanners (IOS) have been developed for mapping and characterizing surface features of the mouth, largely using surface reflectance, including patterned light. The acquired surface contours of dental features enable computer-aided design and fabrication using CAD/CAM (Computer-Aided Design/Computer-Aided Manufacturing) tools.

Dental implants are components used to support some type of dental prosthetic device. Surgically seated within supporting bone structure, the implant integrates into the bone (osseointegration) and then, once securely in place, provides a fixture for mounting the prosthetic device, such as a replacement tooth, bridge, denture, orthodontic fixture, or other element. In many cases, some type of abutment attaches to the dental implant as an intermediary between the implant itself and the prosthetic.

In conventional procedure, implant installation is highly mechanical and has a number of stages, including preparing and drilling an appropriate cavity into the bone surface, seating of the implant within the drilled cavity, osseointegration over a period of weeks to allow the implant to properly affix to the bone tissue, addition of an intermediary abutment, design and fitting of the prosthetic itself, and final fastening and fitting of the prosthesis to the abutment.

Once osseointegration is complete and the implant suitably fixed in place and held by the surrounding bone, the exact angular positioning and orientation of the implant must be precisely determined in order to allow precise design, fabrication, and installation of the prosthetic component. For many types of dental prostheses, intraoral scanning can be used to help determine implant geometry and to facilitate the overall design process. In some cases, a scan body, a temporary positioning component designed and featured to be readily identifiable to scanning software, can be employed to help provide a reference for identifying implant geometry. After a scan of the scan body when it is temporarily installed, the dental implant position can then be detected and used in subsequent computerized processes that advance prosthetics design.

While the scan body can be a practical mechanism for addressing the positioning problem, there remains considerable room for improvement. Because it adds an intermediate device, there is the risk of introducing tolerance and positioning error to the measurement process. Because scan body devices must fit into constrained spaces within the dental arch, their design may not be optimized to facilitate scanning accuracy. It can be challenging to determine scan body position with high levels of confidence, since smooth surfaces and features commonly employed in scan body design can be difficult to detect with sufficient accuracy when installed within the mouth.

It can be appreciated that the use of intermediate devices, such as abutment and scan body components, can introduce some amount of measurement error that can affect final prosthetic positioning. Additional opportunity for human error can be introduced where complex assembly procedures must be performed in order to make measurements. As a result, implant installation can be fairly complex and often requires a high level of training for the practitioner and support staff.

Hand-held IOS devices have been disclosed for use in conventional reflective and video imaging of intraoral features. With conventional devices of this type, there can be some ability for accurate measurement of surface features; however, ability for measurements needed for implant placement typically require accurate measurement of device features that extend below the outer surface. While some IOS devices have been developed to use signals that provide some level of depth-resolved imaging, the widespread use of metal implants (e.g. titanium) renders these devices limited in use. These depth-resolved IOS systems employ non-ionizing energy, using, for example, coherent light, ultrasound, photo-acoustics, or other energy types that can be generated and directed throughout the mouth without concern for radiation exposure to the patient or to the attending staff. These scanning devices have penetrative and non-ionizing attributes that can be highly desirable for measurement of intraoral tissue, teeth, and bones, but have no penetrating ability for metal materials. Thus, to date, such IOS devices have served only for tooth scanning and have not been incorporated for use in implant positioning and fitting.

### SUMMARY OF THE INVENTION

An object of the present disclosure is to advance the art of dental implant prosthesis design and installation using intraoral scanning. Embodiments of the present disclosure particularly address the need for accuracy in determining the position, depth, and axis angle orientation of an installed implant device.

An object of the present disclosure is to address, in whole or in part, at least the foregoing and other deficiencies in the related art. It is a related object of this application to provide, in whole or in part, at least the advantages described herein.

These objects are given only by way of illustrative example, and such objects may be exemplary of one or more embodiments of the application. Other desirable objectives and advantages inherently achieved by the disclosed methods may occur or become apparent to those skilled in the art. The invention is defined by the appended claims.

According to one aspect of the disclosure, there is provided a method comprising:
using a hand-held intraoral scanner, scanning at least a portion of a dental arch having a seated implant and generating a 3-D surface model of the scanned portion of the dental arch from the acquired scan data,
wherein acquired scan data from the intraoral scanner includes both:
   (i) exterior scan data of the scanned portion of the dental arch that characterizes visible external surfaces of the seated implant; and
   (ii) interior scan data that characterizes inner surfaces within a hollow shaft that extends into the implant from an outer opening that is visible on the external surface of the implant to an internal base that terminates the hollow shaft within the implant, wherein the scanned inner surfaces define the orientation of an axis that extends through the hollow shaft;
identifying angular orientation and position of the seated implant within the 3-D surface model according to the axis defined by the interior scan data;
mapping a virtual digital abutment onto the implant in the generated 3-D surface model to form a modified 3-D surface model for the scanned dental arch, according to the identified angular orientation and position;
and
conveying the modified 3-D surface model to a prosthesis design facility.

### BRIEF DESCRIPTION OF DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a schematic diagram that shows the basic anatomy of the conventional dental implant and its relationship to other component parts during installation of a crown or other standard prosthetic device

FIG. 2 is a schematic diagram that shows information needed by the dental lab for forming the prosthesis to suit the position and angulation of the seated implant.

FIG. 3 is a process flow diagram that shows typical steps in the conventional procedure for prosthetic device installation once the implant is fixed in place.

FIG. 4 is a schematic diagram showing basic components of a scanning system at the dental office facility.

FIGS. 5A and 5B are representative images that show, by way of example, how the scan data can be inaccurate and can cause some problems with the process described in FIG. 3.

FIG. 6A is a logic flow diagram that shows a workflow sequence for using the scanner in the prosthesis design process using an embodiment of the present disclosure.

FIG. 6B is a logic flow diagram that shows an alternate workflow sequence for using the scanner in the prosthesis design process using an embodiment of the present disclosure.

FIG. 6C is a logic flow diagram that shows another alternate workflow sequence for using the scanner in the prosthesis design process with the abutment in place, according to an embodiment of the present disclosure.

FIGS. 7A and 7B are perspective views that show scans of implant features using earlier scanners having insufficient depth of field.

FIGS. 8A and 8B are perspective views that show scans of implant features using a hand-held OCT scanner according to an embodiment of the present disclosure.

FIG. 9 is a perspective view showing the use of a virtual abutment placed on a scan of the dental arch.

### DETAILED DESCRIPTION OF THE INVENTION

The following is a detailed description of exemplary embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

In the context of the present disclosure, the term “scattered light” is used generally to include light that is reflected and backscattered from an object.

In the context of the present disclosure, the term “energizable” describes a component or device that is enabled to perform a function upon receiving power and, optionally, upon also receiving an enabling signal.

In the context of the present disclosure, the term “coupled” is intended to indicate a mechanical association, connection, relation, or linking, between two or more components, such that the disposition of one component affects the spatial disposition of a component to which it is coupled. For mechanical coupling, two components need not be in direct contact, but can be linked through one or more intermediary components.

The general term “scanner” relates to an optical system that is energizable to project a scanned beam of light, such as visible light or broadband near-IR (BNIR) light that is directed to the tooth surface. The BNIR light can be directed through a sample arm and acquired, as reflected and scattered light returned in the sample arm, for measuring interference with light from a reference arm used in OCT imaging of a surface. The term “scanner” can also refer to a scanning optical element, such as an actuable MEMS (micro-electromechanical systems) scanner, mirror, or mirror array, for example. The term “raster scanner” relates to the combination of hardware components that sequentially scan light toward uniformly spaced locations along a sample, as described in more detail subsequently.

The term “in signal communication” as used in the application means that two or more devices and/or components are capable of communicating with each other in at least one direction via signals that travel over some type of signal path. Signal communication can be wireless. The term “event” is used herein to indicate an action that causes a signal to be generated, such as an operator press of a scanner button, for example.

The term “subject” refers to the tooth or other portion of a patient that is being imaged and, in optical terms, can be considered equivalent to the “object” of the corresponding imaging system.

Spatially relative terms, such as “beneath”, “below”, “lower”, “above”, “upper”, and the like, may be used herein for ease of description to describe one element or feature's relationship to another element(s) or feature(s) as described and illustrated in the figures. For a patient lying in a dental chair, relative geometry can be applied. Thus, it should be understood that terms that are spatially relative to the scanner and its emitted signals are intended to encompass different orientations of the scanner in use or operation in addition to the orientation depicted in the figures. For scanning within the mouth, for example, scanner orientation must track the local geometry of the intraoral features with relative position considered. Thus, for example, for upper teeth, lesions or objects that can be described herein as lying “beneath” the scanned surface may actually be positioned at a level to, or at a higher absolute spatial elevation in relation to, the scanned surface.

The term “tomography” as used herein relates to reconstruction of surface contour or internal structure of a 3-dimensional (3-D) subject as a “model”, according to information obtained from a penetrating energy. The information that is available for reconstruction can vary with the depth achievable from the penetrating energy.

The term “shaft” as used herein refers to a hollow passageway or channel that is within a dental implant and extends along the implant in a length direction. The shaft can be threaded or un-threaded.

In the context of the present disclosure, the term “characterize” describes the operation of obtaining, representing, or interpreting data about a target feature or object. Scan data, for example, is considered to characterize a surface contour or other aspect of an object.

For hand-held intraoral scanners as described herein, the penetrating energy for imaging is limited to non-ionizing energy, available from visible light, coherent light, ultrasound, photoacoustics, or other lower energy sources scanned into the subject. The IOS generates a 3-D surface model that provides sufficient information on positioning and angular characteristics of the implant that is embedded within the dental art of the patient.

A 3-D surface model, as the term implies, provides a 3-D representation or characterization of the surface contour for a scanned intraoral feature. Once it is generated by stitching together coordinates from adjacent point clouds employing surface points extracted from individual scans, the 3-D surface model is a single-layer characterization of the surface of the intraoral feature, without any image content for tissue that lies beneath the surface.

Apparatus and methods of the present disclosure can provide an IOS solution that simplifies and streamlines the conventional workflow for implant prosthodontics, eliminating the need for using the scan body or other intermediate mechanism in order to measure positioning and alignment of the implant once it is integrated into the dental arch structure.

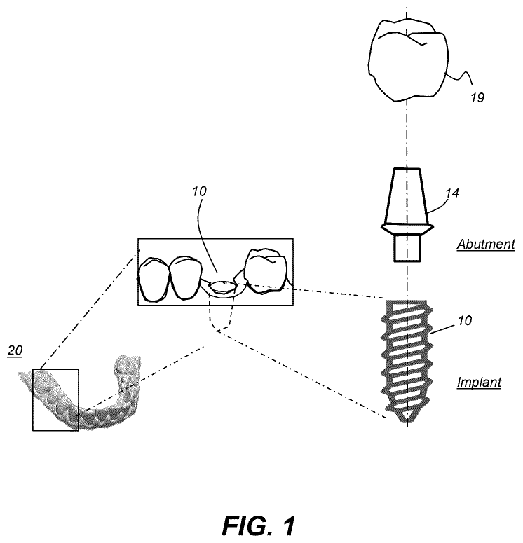

The schematic diagram of FIG. 1 shows the basic anatomy of the conventional dental implant and its relationship to other component parts during installation of a crown or other standard prosthetic device. An implant 10 is fitted into a drilled socket formed within dental arch 20 as part of the initial preparation procedure. The osseointegration process, over a period of weeks, braces implant 10 securely into place, seating implant 10 within the surrounding bone structure so that it is partially exposed or visible and accessible for installation of an abutment 14. Abutment 14 can then be screwed into place or otherwise installed, typically using a fastener (not shown). A prosthetic device 19 can then be fitted onto abutment 14.

Prosthetic device 19 itself, typically built in a dental lab, is designed and formed to be coupled to abutment 14 by implant prosthodontic design software once the actual position and orientation of implant 10 have been determined. A scan body (not shown) can be temporarily fitted onto implant 10 in order to facilitate the determination of implant position and orientation. Scanning of the scan body by an IOS device can then allow the dental lab to determine angular and positional metrics that relate to implant 10 installation so that the final crown or other prosthetic is designed and custom-fitted to the patient's mouth with accuracy.

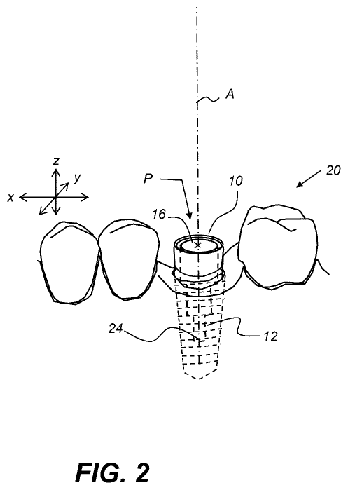

The schematic diagram of FIG. 2 shows information needed by the dental lab for forming the prosthesis to suit the position of the seated implant 10 and angulation of an internal shaft 12 that extends lengthwise through a portion of implant 10, from its terminus at an internal base 24 to an orifice 16 that defines the outer opening of shaft 12. Shaft 12 is hollow, configured to receive a connective fixture for seating the abutment and/or prosthesis and may be threaded, for example. A position P for the implant must be provided; typically, position P relates to the center point coordinates of implant 10 either at the base or at the top of implant 10, such as at an orifice 16, and relative to some benchmark position along the arch. Alternately, position P may give coordinates at the base or along some other point of the implant 10. The angle of an axis A, extending along the length of shaft 12 through the implant orifice, must be determined precisely, in order to properly align and fit the prosthesis. As is well known, point P and a second point along the axis A can be used to define the angular orientation of axis A.

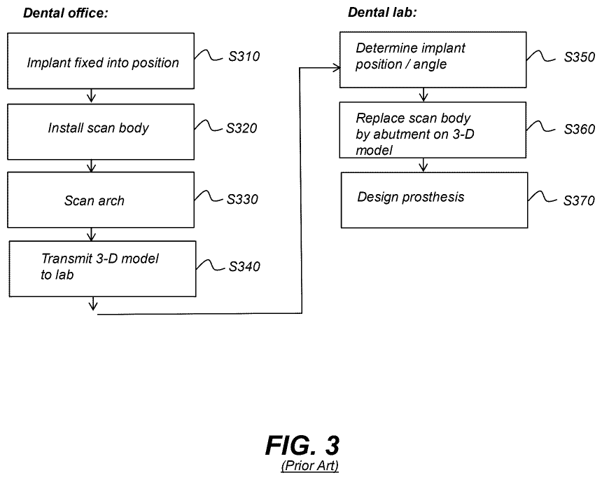

The process flow diagram of FIG. 3 shows typical steps in the conventional procedure for prosthetic device installation once the implant 10 is fixed in place. Steps S310 through S340 are carried out in the dental office; steps S350 through S370 are executed at the dental lab. In an initial step S310, the implant 10 has been fixed in position due to osseointegration. In an install scan body step S320, scan body 18 is fastened in place within implant 10. A scan step S330 is executed, during which the IOS scans dental arch 20. A transmission step S340 executes, in which the 3-D surface model obtained by the IOS is conveyed to the dental lab for prosthesis preparation, typically using internet or other network resources.

At the dental lab, software or other processing is used to determine the relative position and angle of implant 10 based on the scan data in a position determination step S350. Lab software can also execute a replacement step S360 to replace the scan body portion of the obtained 3-D surface model with a virtual abutment, alternately termed a digital abutment. The modified 3-D surface model can then be used for prosthesis design in a design step S370.

As the sequence of FIG. 3 shows, significant coordination can be required between the data acquisition process at the dental office and the modeling and design stages completed at the dental lab. Scan data for the scan body must be accurate in order for replacement of the data to be accurate. Because there are a number of different vendors who supply implant, abutment, and scan body hardware, there are also different software programs employed for this process, making standardization difficult.

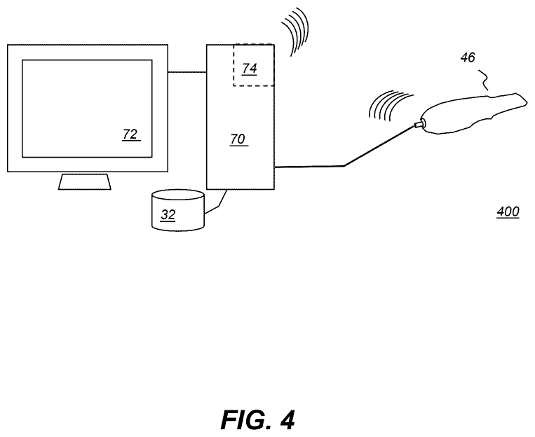

The schematic diagram of FIG. 4 shows basic components of a scanning system 400 at the dental office facility. An intraoral scanner 46 is used by the dental technician or practitioner in order to obtain the scan data. A processor 70 controls operation of scanner 46 and acquires the scan data, which can be stored in a memory 32. Processor 70 can be provided with software for generating a 3-D surface model from the scanned data. A display 72, in signal communication with processor 70, can render a display of the acquired scan and generated 3-D surface model in order to help assist the practitioner to provide useful and complete scan data. As is indicated by a transmitter 74 in FIG. 4, processor 70 can be networked to allow generated 3-D surface model data from the scanner to be transmitted, using wired or wireless communication, or otherwise conveyed to the dental lab facility for prosthesis design and preparation.

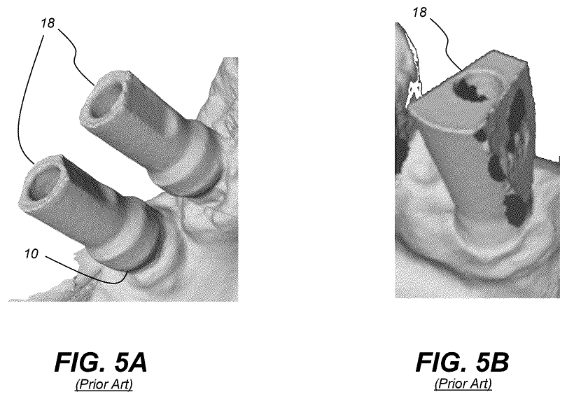

One known problem with the process described with reference to FIG. 3 relates to the reliance on the scanned scan body for providing accurate positional data for the implant. FIGS. 5A and 5B are representative images that show, by way of example, how the scan data for typical scan bodies 18 can be inaccurate and can cause some problems with the process described in FIG. 3. Commonly used scan bodies tend to be highly symmetric in shape with mostly smooth surfaces, making it difficult to scan them well. Scans of symmetric and smooth surfaces can easily confound the stitching process during 3-D model reconstruction, leading to missed data and incorrect matching. This complicates scanning and results in rough or only partially reconstructed scan bodies 18, as shown in the FIG. 5A/5B examples. Even when matching is completed and stitching can be performed, results may be sub-optimal and are prone to errors in scan body reconstruction. As a result, lower mesh quality and errors in reconstructed scan body 18 identification can contribute to less accurate determination of implant position and orientation.

Embodiments of the present disclosure are directed to improvements of the scanning process for determining implant 10 position and orientation and providing the needed information for more efficient and accurate fabrication of the dental prosthesis. Unlike conventional procedures that utilize scans of surrogate or intermediate structures, such as scan body 18, for determining the position and orientation of implant 10, embodiments of the present disclosure directly scan the exposed surface and inner surfaces of implant 10 itself, directing scanning energy into orifice 16 provided by implant 10 and detecting implant angle from the reflected or scattered scan signal that is returned from surfaces within the implant 10 and sensed by the scanner.

Scanning the implant 10 itself can simplify the process of implant prosthesis preparation, bypassing the use of the scan body in the digital implant prosthodontic workflow. The Applicant procedure utilizes an intraoral scanner with a large enough depth-of-field to be able to completely scan both the exposed surfaces and interior of the seated implant. This allows the position and orientation of the implant relative to the dental arch to be directly captured in the full-arch scan. As a result, the need to use the scan body as an implant positioning transfer intermediary is eliminated, thus eliminating the factors that cause implant restoration errors associated with scan body scanning and reconstruction issues. Advantageously, this invention can simplify the workflow needed for implant use, both in the dental office and in the lab.

A suitable intraoral scanner that can scan metal surfaces and deep structures can include an IOS that uses reflective light with triangulation, stereo vision, confocal imaging, or multiple-view capability. Among alternative IOS devices are also scanners normally associated with depth resolved imaging, such as scanners using ultrasound, photoacoustic, or optical coherence tomography (OCT) signals. In general, usable types of hand-held scanners for embodiments of the present disclosure must have a depth-of-field of at least 10 mm, more preferably at least about 15 mm, and must have accurate metal surface scanning capabilities.

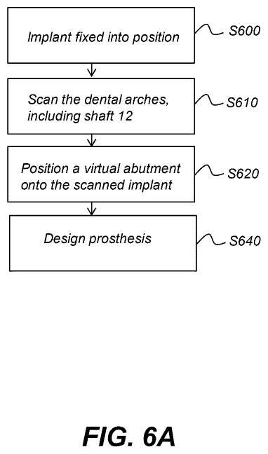

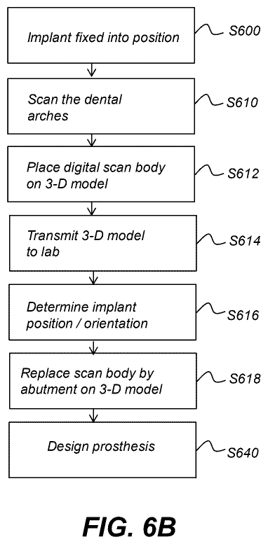

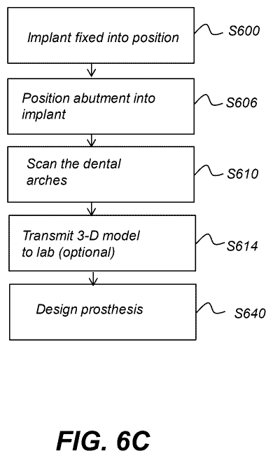

In the Applicant's process, the full arch and the interior of the seated implant 10 are both scanned with an intraoral scanner. Not only is there no step to seat the scan body; the “scan arch” step is simpler and more straightforward due to the absence of scan body components. A workflow sequence for prosthesis design using an embodiment of the present disclosure is shown, for example, in FIG. 6A.

In the FIG. 6A sequence, an initial step S600 includes implant installation and osseointegration, as described previously. Once implant 10 has been securely fixed in place, scan step S610 can be executed, during which the IOS is used to scan some fractional portion or all of the dental arch; this scan can also include data for portions or all of the opposing arch and for the bite. This step can include taking a scan of the dental arch that seamlessly acquires exterior scan data from scanning exposed, visible surfaces of the dental arch and implant 10 and also acquires interior scan data for the hollowed shaft 12, extending from the orifice 16, along interior shaft 12 surfaces of implant 10, and to a base 24 that terminates shaft 12 below the gumline. The scan data from exposed, visible surfaces, also termed exterior scan data herein, characterizes the exposed external surfaces of the dental arch and is useful for showing the relative 3-D position of the implant 10 with respect to neighboring intraoral features and relative to the overall dental arch. The scan data from the interior of implant 10, also termed interior scan data herein, includes scan data that characterizes interior side wall surfaces of shaft 12 that extend from the exposed outer opening of orifice 16 to base 24 within the seated implant 10, generally situated below the gumline. Scan data obtained from base 24 and from the interior surfaces of hollow implant 10 are useful for determining the angular orientation of the implant 10 shaft 12.

Continuing with the FIG. 6A sequence, a digital abutment can be added, mapped to the digital data in an add virtual abutment step S620. In this step, software within the scanner or processor 70 (FIG. 4) or executing remotely (e.g. over the “cloud”) can employ the interior scanned data to compute axis orientation and position for implant 10 and then, using this information, generate and position a virtual abutment 14 for the implant 10 site. Alternately, position and orientation can be determined and used to generate a “best fit” for a virtual abutment 14. According to an embodiment of the present disclosure, the digital abutment 14 can be displayed on display 72 (FIG. 4), or other suitable display, along with scan data, with the virtual abutment 14 integrated as part of the point cloud or surface mesh generated from scanner 46 data and forming a modified 3-D surface model of the arch, for example. Virtually connecting the implant model with a virtual digital abutment or scan body may be achieved by mapping the “connecting part” of the digital abutment or scan body to the inner surface characterization of the implant without a direct need for the angular orientation and position of the implant.

The modified 3-D model can then be used for prosthesis design in a design step S640. The basic workflow sequence of FIG. 6A can be executed at a dental office facility that has an on-site prosthodontic design software and fabrication facility.

In step S620, the digital or virtual abutment 14 is integrated into the 3-D surface model of the scanned arch, providing a mounting feature for design of the crown or other prosthesis. Abutment placement can be accomplished by matching a selected digital abutment to the scanned interior of the implant through algorithms commonly used in computer vision. Algorithms such as iterative closest point (ICP) logic can provide highly accurate results, since no intermediate device is involved as an implant position transferring agent. This approach eliminates tolerancing and other errors, improving the overall accuracy and outcome of prosthesis design. This simplified process can be carried out in the dental office if it is equipped with the implant prosthodontic software for placing digital abutment and designing and manufacturing of the prosthesis.

In one variation of the FIG. 6A sequence, the dental lab provides software tools for placing the digital or virtual abutment into the scanned 3-D model of the dental arches and for the ensuing prosthesis design. In such a case, an additional step (preceding step S620 but not shown in the FIG. 6A sequence) transmits the scanned 3-D model of the dental arches to the dental lab.

At some locations, the dental lab may be reluctant to change the existing workflow process for prosthesis design and preparation. For example, existing vendor software tools might include prosthetic design software that requires the use of a scan body on the scanned dental arch. In such a case, as outlined in FIG. 6B, an additional placement step S612 may be needed for placing a digital, ideal scan body at the implant location in the 3-D model. In this alternate sequence, the modified 3-D model having the digital scan body can be transmitted to the lab in a transmission step S614. In an optional position determination step S616, implant prosthodontic software at the lab operates on the modified 3-D model in the same way as in current workflow to determine the implant position and angular orientation. This can include the use of best-fit techniques for device placement. A replacement step S618 then replaces the virtual scan body with a virtual abutment 14 to allow prosthesis generation in step S640.

The logic flow sequence of FIG. 6C shows an alternative procedure that uses a scan of an actual abutment. As with the sequences described previously, initial step S600 includes implant installation and osseointegration. Once implant 10 has been securely fixed in place, the practitioner executes a position abutment step S606, fastening the physical abutment 14 into implant 10 for scanning the dental arch in scan step S610. The opposing arch and bite can also be scanned. The scanned 3-D model can be used for prosthesis design in step S640 when the dental office that has on-site prosthodontic design software and fabrication facility, or the scanned contents can be transferred to a dental lab facility for processing in optional step S614 and can then be used for prosthesis design in design step S640.

Embodiments of the present disclosure thus allow the dental lab and dental practice to simplify the preparation and planning stages for prosthesis design, while maintaining similar workflow procedures and using existing software.

### IOS Requirements and Types

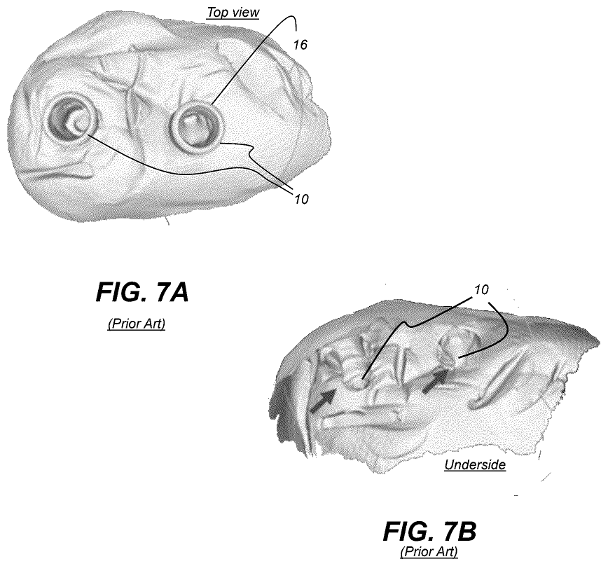

In order to acquire suitable scan content for use in implant preparation, the intraoral scanner (IOS) must have the capability for scanning at sufficient depth of field (DOF). For most dental implant applications, a depth of field (DOF) of at least 15 mm is needed. Earlier IOS devices using reflective light may not have sufficient DOF for implant scanning as described herein. The perspective views of FIGS. 7A and 7B illustrate, by way of example, typical limitations of earlier scanners that fail to have the needed depth imaging capability. As the top view of FIG. 7A shows, the interior surfaces within the scanned implant sites, particularly surfaces below the gumline, may not be completely scanned or clearly distinguishable; as a result, depth reconstruction for implant 10 can be inaccurate, with scanner software attempting to compensate for insufficient scan data by “filling in” the implant orifice, for example. The bottom or underside view of FIG. 7B shows a truncated implant 10 structure due to incomplete scan content.

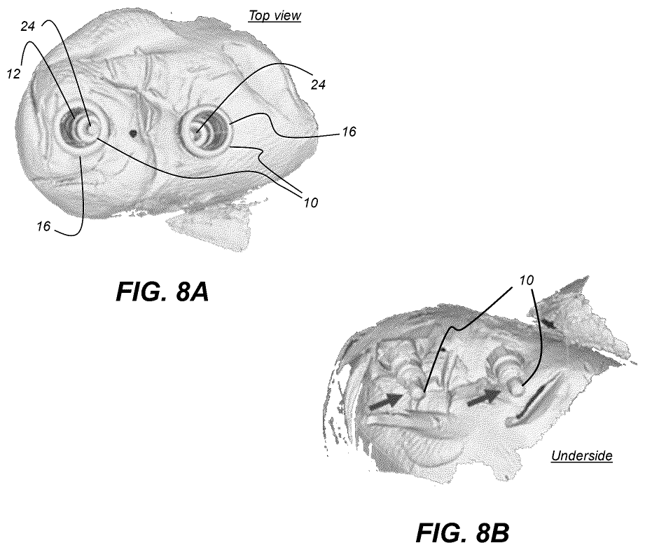

By comparison with FIGS. 7A and 7B, the use of an IOS with suitable DOF and metal scanning capability can obtain a more accurate scan of the interior boundary surfaces of the implant, including surfaces that lie below the gumline, as shown in the examples of FIGS. 8A and 8B. In these examples, internal features of implant 10 can be more clearly scanned and rendered, with a more accurate reconstruction of the base 24 of implant 10 within the arch.

Recent improvements in triangularization and optics may allow sufficient DOF scanning using reflective light in some embodiments of the present disclosure. Improved triangularization, for example, can allow a reflective imaging system to reconstruct the inner surface of implant 10 with enough accuracy for determining axis A position and angular orientation. Triangulation or confocal imaging approaches can help to provide the needed DOF, particularly considering width constraints for the implant 10 orifice. Improved stereo vision and multi-view capability can also yield higher DOF values for the IOS.

### OCT Advantages

Intraoral scanners using Optical Coherence Tomography (OCT) can also provide sufficient DOF and resolution for scanning exposed implant 10 surfaces, including surfaces internal to the implant 10 orifice and surfaces that extend outside the gum tissue. Because it senses interference effects of a coherent light signal, the OCT scanner can have a large DOF, sufficient for penetrating fully within the orifice of implant 10. Additionally, OCT-based IOS do not encounter signal degradation when scanning reflective surfaces and are, therefore, capable to scan dental materials made of metal, such as implants, completely and accurately.

As described with reference to FIG. 4, processor 70 associated with the OCT scanner or other type of IOS can be provided with software for generating a 3-D surface model using the scanned data. Reconstruction of the 3-D surface model of the dental arch includes at least exposed portions of implant 10 and includes data that precisely locates the position of orifice 16 relative to the dental arch. Axis A extends through the center of orifice 16. As described previously with regard to FIG. 2, the angle of axis A relative to the dental arch can be determined by two points, such as a position P and some other point. The center of orifice 16 can provide one point, for example. Some other point within orifice 16 can serve to define axis A or to define a vector that is parallel to axis A. Because the dimensions and features of the implant 10 itself are known, identifying a feature such as a point at the very bottom of the implant 10 can be used to help determine axis A angle. An alternate method for determining the angle of axis A is to construct a line normal to the plane of the top surface of implant 10 and extending through the center of orifice 16.

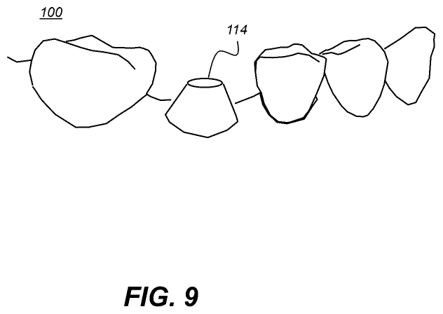

FIG. 9 is a perspective view showing the use of a virtual abutment 114 placed on a 3-D surface model from the scan of a dental arch 100. The digital abutment 114 can be a 3-D feature added to the 3-D surface model by processor 70 software or by some other processor in the office or dental lab facility or remote processor. Virtual abutment 114 can be provided from a stored library of abutment devices, arranged by implant manufacturer, for example.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of invention is indicated by the appended claims, and all changes that come within the meaning and range of equivalents thereof are intended to be embraced therein.

1. A method comprising:
using a hand-held intraoral scanner, scanning at least a portion of a dental arch having a seated implant and generating a 3-D surface model of the scanned portion of the dental arch from the acquired scan data,
wherein acquired scan data from the intraoral scanner includes both:
(i) exterior scan data of the scanned portion of the dental arch that characterizes visible external surfaces of the seated implant; and
(ii) interior scan data that characterizes inner surfaces within a hollow shaft that extends into the implant from an outer opening that is visible on the external surface of the implant to an internal base that terminates the hollow shaft within the implant, wherein the scanned inner surfaces define the orientation of an axis that extends through the hollow shaft;
mapping a virtual digital abutment onto the implant in the generated 3-D surface model to form a modified 3-D surface model for the scanned dental arch, according to the inner surface characterization of the implant in the 3-D surface model;
and
conveying the modified 3-D surface model to a prosthesis design facility.

2. The method of claim 1 wherein the prosthesis design facility is on-site or on a remote computer.

3. The method of claim 1 wherein the 3-D surface model comprises surface contour for the scanned portion of the dental arch.

4. The method of claim 1 wherein the 3-D surface model further comprises at least a portion of an opposing arch and a bite model.

5. The method of claim 1 wherein the hand-held intraoral scanner is an OCT scanner.

6. The method of claim 1 wherein the hand-held intraoral scanner directs light to the scanned surface.

7. The method of claim 1 wherein the hand-held intraoral scanner is an ultrasound scanner.

8. The method of claim 1 wherein the hand-held intraoral scanner is a photoacoustic scanner.

9. The method of claim 1 further comprising identifying angular orientation and position of the seated implant within the 3-D surface model according to the axis defined by the interior scan data.

10. The method of claim 1 wherein the hand-held intraoral scanner uses triangulation or confocal imaging.

11. A method for dental implant prosthesis design, comprising:
scanning a portion of a dental arch having a seated implant using a hand-held intraoral scanner, wherein the acquired intraoral scan data characterizes visible external surfaces of the seated implant and further characterizes the inner surface of a hollow shaft of the implant, the hollow shaft extending from an outer opening to an internal base that terminates the hollow shaft;
generating a 3-D surface model of the scanned portion of the dental arch from acquired intraoral scan data;
mapping a digital scan body onto the scanned implant to form a first modified 3-D surface model, according to the inner surface characterization of the implant in the 3-D surface model;
transmitting the first modified 3-D surface model to a prosthesis design facility;
replacing the digital scan body on the first modified 3-D surface model with a virtual digital abutment to form a second modified 3-D surface model, according to the position of the digital scan body;
and
performing prosthesis design using the second modified 3-D surface model.

12. The method of claim 11 further comprising determining the position and orientation of the seated implant according to the digital scan body on the first modified 3-D surface model.

13. An apparatus for dental implant prosthesis design, comprising:
a hand-held intraoral scanner configured to scan a seated implant in a dental arch and to acquire both:
(i) exterior scan data of a scanned portion of the dental arch that characterizes visible external surfaces of the seated implant; and
(ii) interior scan data that characterizes inner surfaces within a hollow shaft that extends into the implant from an outer opening that is visible on the external surface of the implant to an internal base that terminates the hollow shaft within the implant, wherein the scanned inner surfaces define the orientation of an axis that extends through the hollow shaft;
a processor in signal communication with the scanner and configured to generate a 3-D surface model of the scanned implant within the scanned portion of the dental arch;
and
a transmitter in signal communication with the processor and configured to transmit at least the generated 3-D surface model to a prosthesis design facility.

14. The apparatus of claim 13 wherein the prosthesis design facility is on-site or on a remote computer.

15. The apparatus of claim 13 wherein the hand-held intraoral scanner is an OCT scanner.

16. The apparatus of claim 13 wherein the hand-held intraoral scanner directs light to the scanned surface.

17. The apparatus of method 13 wherein the hand-held intraoral scanner has a depth of field of at least 10 mm.

18. The apparatus of claim 13 wherein angular orientation and position of the seated implant are modified according to an axis that is defined by interior scan data.

19. The apparatus of claim 13 wherein the hand-held intraoral scanner is a triangulation or confocal imaging system.

20. The apparatus of claim 13 wherein the processor is further configured to map a virtual digital abutment onto the implant in the generated 3-D surface model to form a modified 3-D surface model for the scanned dental arch, according to inner surface characterization for the implant.
